(19) United States
(12) Patent Application Publication
(10) Pub. No.: US 2024/0279271 A1
(43) Pub. Date: Aug. 22, 2024

(54) Method To Purify An Antibody Composition Using Cation Exchange Chromatography

(71) Applicant: DR. REDDY’S LABORATORIES LIMITED, Hyderabad (IN)

(72) Inventors: Ram KUMAR M., Krishnagiri District (IN); Roja APUORVA T., Dharmapuri (IN); Kishore JAHAGIRDAR, Hyderabad (IN)

(21) Appl. No.: 18/681,162

(22) PCT Filed: Aug. 4, 2022
(86) PCT No.: PCT/IN2022/050701

§ 371 Date: Feb. 5, 2024

(30) Foreign Application Priority Data
Aug. 5, 2021 (IN) 202141035332

(51) Int. Cl. C07K 1/18; C07K 16/28

(52) U.S. Cl. CPC C07K 1/18; C07K 16/2839

(57) ABSTRACT

The method disclosed in the current invention is used to purify an antibody from process and product related impurities. The method discloses the use of cation exchange chromatography for the reduction of impurities such as high molecular weight aggregates, protein-A leachates and host cell proteins from an antibody composition. The disclosed method leads to a significant reduction of HMW aggregates and other process related impurities without compromising on the recovery of the protein.

### FIELD OF THE INVENTION

The present invention relates to protein purification methods. In particular, the invention relates to methods for purifying an antibody composition using ion exchange chromatography.

### BACKGROUND

Monoclonal antibodies (mAbs) are effective targeted therapeutic agents. The high specificity of the antibodies makes them ideal to reach their intended target and hence is useful to treat a wide variety of diseases.

The commercial production of recombinant human monoclonal antibody therapeutics demands robust processes, i.e., the purification scheme needs to reliably and predictably produce antibody composition intended for use in humans. A process should be designed to remove the product related contaminants such as high molecular weight (HMW) aggregates, product variants such as charged variants (acidic, deamidated/oxidized, basic), sequence variants and other species, as well as process related contaminants such as leached Protein-A, host cell protein, DNA, adventitious and endogenous viruses, endotoxin, extractable from resins and filters, process buffers and agents such as detergents that may have been employed for virus reduction. In designing a purification scheme and other conditions for each of the chromatographic steps, along with removal of contaminants, an important consideration is recovery from each step of the purification scheme and from the overall purification scheme. Hence, for a commercially viable process, the purification scheme needs to be designed to ensure adequate removal of contaminants from an antibody composition while maintaining the yield of the same.

Chromatographic techniques exploit the physical and chemical differences between the antibodies and the contaminant for the separation. Majority of purification schemes for mAbs involve a Protein-A based chromatography, which results in a high degree of purity and recovery in a single step. One or two additional chromatography steps are employed as polishing steps, generally selected from cation and anion exchange chromatography, although hydrophobic interaction chromatography, mixed mode chromatography or hydroxyapatite chromatography may be chosen as well.

Removal of aggregates, especially soluble aggregates, presents a challenge due to the physical and chemical similarity of the aggregates to the drug product itself, which is usually a monomer. This challenge becomes even more daunting when the antibody to be purified is hydrophobic in nature, such as an anti-α4β7 antibody, for example, vedolizumab. Cation exchange chromatography (CEX), hydrophobic interaction chromatography (HIC), mixed-mode chromatography (MMC) or combinations thereof are generally used to control the HMW aggregates of antibodies. However, controlling of level of HMW aggregates becomes increasingly difficult if the process is devoid of chromatographic steps such as HIC and MMC and, specifically if the formation of HMW increases at any stage during the chromatographic or purification process.

Hence, there is a need for an improved process to control the HMW aggregates and product and process related impurities in the final drug substance of a therapeutic antibody composition.

### SUMMARY

The present invention discloses a method for purifying an antibody composition comprising the antibody and one or more contaminants by contacting the said antibody composition with a cation exchange support under conditions in which the antibody substantially binds to the support, washing the cation exchange support with a wash buffer, passing an elution buffer through the cation exchange support and collecting the eluate from the cation exchange support, wherein the elution buffer has a pH of about 6 and/or conductivity of less than 10 mS/cm, and the mode of elution is gradient elution.

The specific elution conditions employed effect >80% reduction of HMW aggregates, >90% reduction of protein-A leachates and greater than 10-fold reduction (1 log reduction) in the HCP content, further maintaining the recovery of the antibody to be about 90% or more.

The method disclosed as per the current invention is advantageous as it may not require further chromatographic steps such as HIC or MMC for the reduction of HMW aggregates.

### DETAILED DESCRIPTION OF THE INVENTION

### Definitions

The phrase “ion exchange material” refers to a solid phase which is negatively charged (i.e., a cation exchange resin) or positively charged (i.e., an anion exchange resin). The charge may be provided by attaching one or more charged ligands to the solid phase, e.g. by covalent linking. Alternatively, or in addition, the charge may be an inherent property of the solid phase (e.g. as is the case for silica, which has an overall negative charge).

The term “conductivity” refers to the ability of an aqueous solution to conduct an electric current between two electrodes. In solution, the current flows by ion transport. Therefore, with an increasing amount of ions present in the aqueous solution, the solution will have a higher conductivity. The unit of measurement for conductivity is mS/cm, and can be measured using a conductivity meter, e.g., by Orion. The conductivity of a solution may be altered by changing the concentration of ions therein. For example, the concentration of a buffering agent and/or concentration of a salt (e.g. NaCl or KCl) in the solution may be altered in order to achieve the desired conductivity.

A “contaminant” is a material that is different from the desired polypeptide product. The contaminant may be a variant of the desired polypeptide (e.g. a deamidated variant or an aminoaspartate variant of the desired polypeptide) or another non-product related polypeptide, for e.g., host cell protein, host cell nucleic acid, endotoxin, etc. A contaminant can also be process related, for example—Protein-A-leachates.

“High molecular weight aggregates” as referred herein encompasses association of at least two molecules of a product of interest, e.g., antibody or any antigen-binding fragment thereof. The association of at least two molecules of a product of interest may arise by any means including, but not limited to, non-covalent interactions such as, e.g., charge-charge, hydrophobic and van der Waals interactions; and covalent interactions such as, e.g., disulfide interaction or non-reducible crosslinking. An aggregate can be a dimer, trimer, tetramer, or a multimer greater than a tetramer, etc.

The term “process or product related impurities” as used herein refer to the contaminants which may be derived from the manufacturing process, for example, but not limited to, cell culture, downstream or cell substrates and may include host cell proteins, host cell DNA, nucleic acid, protein-A leachates etc., or may be molecular variants of the protein of interest, for example, but not limited to, HMW aggregates, acidic variants, basic variants, low molecular weight variants etc., and may be formed during expression, manufacture or storage of the protein.

The term “about” as used herein, would mean and include a variation of up to 10% from the particular value.

The “composition” to be purified herein comprises the protein of interest and one or more contaminants. The composition may be “partially purified” (i.e., having been subjected to one or more purification steps) or may be obtained directly from a host cell or organism producing the antibody (e.g., the composition may comprise harvested cell culture fluid).

The term “load” herein refers to the composition loaded onto the chromatography material, i.e., ion exchange support. Preferably, the chromatography material is equilibrated with an equilibration buffer prior to loading the composition which is to be purified.

The term “bind and elute mode” as used herein refers to a process wherein the target protein substantially binds to the chromatographic support, and is subsequently eluted from the chromatographic support.

Aggregate concentration can be measured in a protein sample using Size Exclusion Chromatography (SEC), a well-known and widely accepted method in the art. Size exclusion chromatography uses a molecular sieving retention mechanism, based on differences in the hydrodynamic radii or differences in size of proteins. Large molecular weight aggregates cannot penetrate or only partially penetrate the pores of the stationary phase. Hence, the larger aggregates elute first and smaller molecules elute later, the order of elution being a function of the size.

### DETAILED DESCRIPTION OF THE EMBODIMENTS

The present invention discloses a method to purify an antibody composition comprising the antibody and contaminants, for example, high molecular weight aggregates, host cell proteins/nucleic acids, protein-A leachates, the method comprises the use of cation exchange chromatography.

In an embodiment, the method is used to reduce the level of process and product related impurities in an antibody composition comprising an antibody and one or more said impurities using cation exchange chromatography.

In another embodiment, the method is used to reduce the level of process and product related impurities in an antibody composition comprising an antibody and one or more said impurities using cation exchange chromatography, wherein the antibody composition is contacted with the cation exchange support in the presence of a loading buffer solution under such conditions that the antibody substantially binds to the cation exchange support, and the bound antibody is eluted from the cation exchange support by a gradient elution using an elution buffer at a pH of about 6.

In another embodiment, the method disclosed in the invention is used to reduce the level of process and product related impurities in an antibody composition comprising an antibody and one or more said impurities, the method comprising steps of:
(a) contacting the antibody composition with a cation exchange support in the presence of a loading buffer solution under conditions such that the antibody substantially binds to the cation exchange support,
(b) optionally washing the cation exchange support with a wash buffer solution,
(c) eluting the bound antibody by gradient elution using an elution buffer, and
(d) collecting the eluate from the cation exchange support, wherein the elution buffer solution has a pH of about 6.

In another embodiment, the method disclosed in the invention is used to reduce the level of process and product related impurities in an antibody composition comprising an antibody and one or more said impurities, the method comprising steps of:
(a) contacting the antibody composition with a cation exchange support in the presence of a loading buffer solution under conditions such that the antibody substantially binds to the cation exchange support,
(b) optionally washing the cation exchange support with a wash buffer solution,
(c) eluting the bound antibody by gradient elution using an elution buffer, and
(d) collecting the eluate from the cation exchange support, wherein the elution buffer solution has a conductivity of less than 10 mS/cm.

In another embodiment, the method disclosed in the invention is used to reduce the level of process and product related impurities in an antibody composition comprising an antibody and one or more said impurities, the method comprising steps of:
(a) contacting the antibody composition with a cation exchange support in the presence of a loading buffer solution under conditions such that the antibody substantially binds to the cation exchange support,
(b) optionally washing the cation exchange support with a wash buffer solution,
(c) eluting the bound antibody by gradient elution using an elution buffer, and
(d) collecting the eluate from the cation exchange support, wherein the elution buffer solution has a pH of about 6 and conductivity of less than 10 mS/cm.

In any of the above mentioned embodiments, the high molecular weight aggregates are reduced by at least 80% in the eluate collected from the cation exchange support as compared to the level of high molecular weight aggregates in the antibody composition loaded onto the cation exchange support.

In any of the above mentioned embodiments, the method is also used to reduce the level of other process-related impurities, including but not limited to protein-A leachates, host cell proteins, host cell DNA, etc.

In any of the above mentioned embodiments, the method is used to reduce the level of HCPs in the antibody composition by more 10-folds.

In any of the above mentioned embodiments, the amount of host cell DNA in the eluate is about 1 pg/mg.

In any of the above mentioned embodiments, the recovery of the antibody in the eluate is not less than 90%.

In any of the above mentioned embodiments, the recovery of the antibody in the eluate is about 92% or more.

In any of the above mentioned embodiments, the CEX is operated in bind and elute mode.

In any of the above mentioned embodiments, the loading buffer solution is phosphate buffer.

In any of the above mentioned embodiments, the pH and conductivity of the wash buffer solution is same as that of the loading buffer solution.

In any of the above mentioned embodiments, the pH and conductivity of the wash buffer solution is different than that of the loading buffer solution.

In any of the above mentioned embodiments, the elution buffer comprises a gradient of two buffer solutions: elution buffer A and elution buffer B.

In the above mentioned embodiment, elution buffer A is 50 mM phosphate buffer and elution buffer B is 50 mM phosphate buffer with 250 mM NaCl.

In the above mentioned embodiment, pH of elution buffer A is 5.9 and conductivity is about 4 mS/cm.

In the above mentioned embodiment, pH of elution buffer B is 5.9 and conductivity is about 26 mS/cm.

In any of the above mentioned embodiments, the conductivity of the elution buffer solution at any given point of time during the gradient is less than 10 mS/cm.

In any of the above mentioned embodiments, the conductivity of the elution buffer solution is in the range of 4 mS/cm to 10 mS/cm.

In any of the above mentioned embodiments, the conductivity of the elution buffer solution is in the range of 6 mS/to 10 mS/cm

In any of the above mentioned embodiments, the conductivity of the elution buffer solution is in the range of 8 mS/cm to 10 mS/cm.

In any of the above mentioned embodiments, the antibody is an anti-α4β7 antibody or antigen binding fragment thereof.

In any of the above mentioned embodiments, the antibody is vedolizumab.

The invention is more fully understood by reference to the following examples. These examples should not, however, be construed as limiting the scope of the invention.

### EXAMPLES

### Example 1

A therapeutic monoclonal antibody which binds to human α4β7 integrin was cloned and expressed in a Chinese Hamster Ovary cell line and the cell culture broth containing the expressed antibody was harvested, clarified and subjected to protein-A affinity chromatography. The process was carried out initially at a 50-liter scale and then it was scaled up to 1000-liters. The eluate from protein-A affinity chromatography was subjected to low-pH incubation and depth filtration, and the filtered liquid comprising the antibody composition was further purified using cation exchange chromatography (CEX). Level of impurities was determined in both load and eluate of CEX. Details of CEX chromatography are given in Tables 1 and 2. It is to be noted that samples Vmab-1, Vmab-2 and Vmab-3 were processed at the 50-liter scale, whereas samples Vmab-4, Vmab-5 and Vmab-6 were processed at the 1000-liter scale.

TABLE 1
Chromatography conditions used in CEX

| Resin | Poros ™ XS |
|---|---|
| Bed Height (cm) | 18-22 |
| Residence Time (min) (loading, post- | 4.0 |
| load wash and elution) | |
| Load Factor (g/L) | 40 |

TABLE 2
Details of buffers used in CEX

| | | | Conductivity |
|---|---|---|---|
| Stage | Buffer | pH | (mS/cm) |
| Equilibration | 50 mM phosphate buffer | 5.9 | 4 ± 1 |
| Wash | 50 mM phosphate buffer | 5.9 | 4 ± 1 |
| Elution | Linear gradient, 0%-30% B in 15 CV | 5.9 | A: 4 ± 1 |
| | A: 50 mM phosphate buffer, | | B: 26 ± 2 |
| | B: 50 mM phosphate buffer, | | |
| | 250 mM NaCl | | |
| Regeneration | 0.5M NaOH | NA | NA |

Tables 3 and 4 summarize the HMW aggregate level at the time of loading onto CEX and in the eluate obtained from CEX at elution buffer pH of 5.9 at 50 L scale and 1000 L scale, respectively. Table 3 and 4 also summarizes the recovery % obtained at 50 L and 1000 L scale respectively post CEX chromatography.

TABLE 3
HMW aggregate level in CEX load
and CEX eluate from 50 L scale

| Batch | HMW % (load) | HMW % (eluate) | % Reduction | % Recovery |
|---|---|---|---|---|
| Vmab-1 | 1.5 | 0.3 | 80.0% | 91 |
| Vmab-2 | 1.6 | 0.3 | 81.3% | 94 |
| Vmab-3 | 1.4 | 0.3 | 78.6% | 100 |

TABLE 4
HMW aggregate level in CEX load and CEX eluate from 1000 L scale

| | HMW | HMW % | | Avg. | | |
|---|---|---|---|---|---|---|
| | % | (eluate) | | HMW % | % | % |
| Batch | (load) | Lot 1 | Lot 2 | (eluate) | Reduction | recovery |
| Vmab_4 | 1.2 | 0.20 | 0.30 | 0.25 | 79.2% | 92.9 |
| Vmab-5 | 1.2 | 0.30 | 0.30 | 0.30 | 75.0% | 92.9 |
| Vmab-6 | 1.2 | 0.20 | 0.20 | 0.20 | 83.3% | 94.8 |

Similarly, the levels of HCP and protein-A leachates were determined in CEX load and eluate and are represented in Table 5 and 6 along with HCD content in CEX eluate.

TABLE 5
HCP and PAL levels at CEX load and CEX eluate at 50 L scale

| | HCP (ng/mg) | | PAL (ng/mg) | | |
|---|---|---|---|---|---|
| | CEX | CEX | CEX | CEX | HCD (pg/mg) |
| Batch number | load | eluate | load | eluate | CEX eluate |
| Vmab-1 | 170 | 4.4 | 14.0 | 1.6 | 1.3 |
| Vmab-2 | 194 | 4.6 | 16.0 | 2.2 | 0.2 |
| Vmab-3 | 173 | 4.1 | 16.5 | 1.2 | 0.8 |

TABLE 6
Table 7: HCP and PAL levels at CEX
load and CEX eluate at 50 L scale

| | HCP (ng/mg) | | | PAL (ng/mg) | | | HCD (pg/mg) | |
|---|---|---|---|---|---|---|---|---|
| Batch | CEX | CEX eluate | | CEX | CEX eluate | | CEX eluate | |
| number | load | Lot 1 | Lot 2 | load | Lot 1 | Lot 2 | Lot 1 | Lot 2 |
| Vmab-4 | 237.3 | 5.0 | 4.9 | NA | <1.15 | <1.18 | <1.12 | <1.14 |
| Vmab-5 | 264.3 | 4.8 | 4.8 | NA | <1.04 | <1.14 | <1.00 | <1.11 |

It is evident from Tables 3-6 that the disclosed method is able to significantly reduce the levels of HMW aggregate in addition to the removal of HCP and protein-A leachates.

A full factorial design of experiments (DOE) study was carried out to investigate the effect of elution buffer pH and load factor on the HMW aggregate reduction. Table 5 lists the elution buffer pH, load factor, HMW aggregate percentage and percentage recovery in the CEX eluate.

TABLE 8
% HMW aggregate and % recovery values at different
elution buffer pH and load factor

| | Elution | Load | % HMW | |
|---|---|---|---|---|
| S. No | buffer pH | factor (g/L) | (eluate) | % Recovery |
| 1 | 5.7 | 40 | 0.21 | 91.2 |
| 2 | 5.7 | 40 | 0.21 | 92.7 |
| 3 | 5.7 | 40 | 0.21 | 91.5 |
| 4 | 5.7 | 40 | 0.21 | 91.2 |
| 5 | 6.1 | 40 | 0.62 | 94.8 |
| 6 | 6.1 | 40 | 0.61 | 94.5 |
| 7 | 6.1 | 40 | 0.59 | 94.4 |

From Table 7, it is clear that although pH 5.7 results in the minimum HMW aggregates in the eluate, it also leads to lower recovery of the protein and a much higher elution volume (data not shown) for the target antibody, which leads to a longer process time and thus is not economically advantageous. Next, an elution buffer pH of 6.1 leads to higher HMW aggregates in the eluate, which is not desirable. Therefore, at a load factor of 40 g/L, elution buffer pH of 5.9 and a conductivity of below 10 mS/cm is the most advantageous, both in terms of reduction of HMW aggregates and in terms of process economics.

1. A method to reduce the level of process and product related impurities in an anti-α4β7 antibody composition comprising an anti-α4β7 antibody and one or more said impurities, the method comprising steps of:
(a) contacting the antibody composition with a cation exchange support in the presence of a loading buffer solution under conditions such that the antibody substantially binds to the cation exchange support,
(b) optionally washing the cation exchange support with a wash buffer solution,
(c) eluting the bound antibody by gradient elution using an elution buffer, and
(d) collecting the eluate from the cation exchange support,
wherein the elution buffer solution has a pH of about 6 and conductivity of less than 10 mS/cm.

2. The method as claimed in claim 1, wherein the loading buffer solution comprises phosphate.

3. The method as claimed in claim 1, wherein the elution buffer comprises a gradient of two buffer solutions, elution buffer A and elution buffer B, and wherein elution buffer A comprises 50 mM phosphate and elution buffer B comprises 50 mM phosphate and 250 mM sodium chloride.

4. The method as claimed in claim 3, wherein elution buffer A has a pH of 5.9 and conductivity of about 4 mS/cm and elution buffer B has a pH of 5.9 and conductivity of about 26 mS/cm.

5. The method as claimed in claim 1, wherein the conductivity of the elution buffer solution at any given point of time during the gradient is between 4 mS/cm and 10 mS/cm.

6. The method as claimed in claim 1, wherein the anti-α4β7 antibody is vedolizumab.

7. The method as claimed in claim 1, wherein the anti-α4β7 antibody collected as an eluate from the cation exchange support has a recovery of about 92% or more.

8. The method as claimed in claim 1, wherein the impurities are selected from high-molecular weight aggregates, antibody fragments, host cell proteins, host cell DNA, protein-A leachates, endotoxins, acidic variants and basic variants.

9. The method as claimed in claim 8, wherein the high-molecular weight aggregates are reduced by at least 80% in the eluate collected from the cation exchange support as compared to the level of high molecular weight aggregates in the antibody composition loaded onto the cation exchange support.

10. The method as claimed in claim 1, wherein the level of host cell proteins is reduced by more than 10-folds in the eluate collected from the cation exchange support as compared to the level of host cell proteins in the antibody composition loaded onto the cation exchange support.
